(19) United States
(12) Patent Application Publication
(10) Pub. No.: US 2022/0125834 A1
(43) Pub. Date: Apr. 28, 2022

(54) LOCALIZED DELIVERY OF THERAPEUTIC AGENTS

(71) Applicant: Nostopharma, LLC, Bethesda, MD (US)

(72) Inventor: Ekaterina VERT-WONG, Potomac, MD (US)

(21) Appl. No.: 17/438,195

(22) PCT Filed: Mar. 10, 2020
(86) PCT No.: PCT/US2020/021900

§ 371 Date: Sep. 10, 2021

Related U.S. Application Data
(60) Provisional application No. 62/817,531, filed on Mar. 12, 2019.
(60) Provisional application No. 62/939,439, filed on Nov. 22, 2019.

(51) Int. Cl. A61K 33/36; A61K 31/593; A61K 45/06; A61K 9/00

(52) U.S. Cl. CPC A61K 33/36; A61K 9/0024; A61K 45/06; A61K 31/593

(57) ABSTRACT

The present invention is directed to compositions and methods for the prevention or treatment of diseases or conditions, including heterotopic ossification, vascular calcification, or pathologic calcification involving methods of drug delivery that allow soft tissue to be treated without interfering with normal processes of bone formation or calcification.

### CROSS REFERENCE TO RELATED APPLICATIONS

The present application claims the benefit of U.S. provisional patent application No. 62/939,439, filed on Nov. 22, 2019 and the benefit of U.S. provisional patent application No. 62/817,531 filed on Mar. 12, 2019.

### FIELD OF THE INVENTION

The present invention is directed to methods of therapeutically administering drugs or drug combinations to different anatomic sites. In an especially preferred embodiment, a combination of two or more therapeutic agents selected from the group consisting of a Hedgehog signaling pathway antagonist; vitamin D, cholecalciferol or a vitamin D analog; and/or a statin is used together with one or more other drugs.

### BACKGROUND OF THE INVENTION

Heterotopic ossification is bone growth in tissues where ossification should not be occurring. Abnormal growth may occur in response to trauma, burn, inflammation, autoimmune attack or other types of tissue disruption. Analyses have suggested that the cellular origin for ectopic bone formation may be mesenchymal progenitor cells. The differentiation of these cells into osteogenic lineages is induced by a pathological microenvironment in soft tissues outside the skeletal tissue, which includes inflammation.

It has recently been reported that drug combinations of: Hedgehog signaling pathway antagonists; vitamin D, cholecalciferol or a vitamin D analog; and/or statins can be used in preventing and treating heterotopic ossification, vascular calcification, and pathologic calcification (US 2018/0071319). This treatment may be used to treat ectopic bone formation or calcification of any origin, including spinal cord damage, traumatic injuries, head and brain injuries, burns, bone fractures, muscle injuries, and surgery. It should also be effective in patients with diseases or conditions that predispose them to ectopic bone formation or calcification, such as atherosclerosis or myocardial infarction, chronic inflammation, such as ankylosing spondylitis (a type of arthritis that mostly affects the lower part of the spine, and where it joins to the hips, known as the sacroiliac joints) and other complications of autoimmune conditions, perimyositis (inflammation of connective tissue around a muscle), and in genetic diseases such as osseous heteroplasia, fibrodysplasia ossificans progressiva, Albright's hereditary osteodystrophy and osteopetrosis.

However, in cases where broken bones are present in a patient, inhibition of new bone formation may slow the healing process. Given that bone and soft tissue around it (muscles, nerve channels, ligaments, joint tissues) lie in close proximity to one another, it is important to develop a treatment modality that maintains the concentration and action of the active ingredients in blocking bone formation in soft tissues but that does not substantially inhibit normal healing processes at the site of breakage. The same basic devices and procedures described herein may be used whenever it is desirable to confine locally delivered drugs to a specific bodily site and particularly when it is advantageous to deliver one drug to a first site and a different drug to a different site in close proximity to the first.

### SUMMARY OF THE INVENTION

### A. General Summary

The present invention is based, in part, on the concept that it is possible to prevent or treat heterotopic ossification, vascular calcification, or other pathologic calcification using the compounds and combinations described in US 2018/0071319 (and also U.S. Pat. No. 10,456,409) and, at the same time, maintain normal processes involving bone formation or calcification. Thus, in cases where a patient has broken bones or trauma from orthopedic surgery, heterotopic ossification and calcification may be treated or prevented without preventing bone healing. This is accomplished by using drug delivery methods that localize therapeutically effective concentrations at sites where treatment or prevention are needed but that produce much lower levels at other sites. The invention also extends to situations where cartilage repair is needed in a patient and methods of preventing or treating pathologic ossification or calcification may be beneficial. Apart from this, the invention more generally includes any treatment method in which it is desirable to locally treat a disease or condition but confine drug delivery to a specific anatomic area so as to avoid one or more unwanted side effects at nearby anatomic sites.

### B. Summary of Specific Embodiments

Method for Inhibiting Osteogenesis in Mesenchymal Cells

In a first aspect, the invention is directed to a method for inhibiting osteogenesis in mesenchymal stem cells in a patient, comprising contacting the mesenchymal cells with a drug selected from the group consisting of: a Hedgehog (Hh) pathway antagonist; vitamin D; cholecalciferol; a vitamin D analog; and a statin; or a combination of drugs selected from:
a) a combination of a Hedgehog (Hh) pathway antagonist together with:
   i) vitamin D, cholecalciferol or a vitamin D analog; or
   ii) a statin;
b) a combination of:
   i) vitamin D, cholecalciferol or a vitamin D analog; and
   ii) a statin; or
c) a combination of:
   i) an Hh pathway antagonist;
   ii) vitamin D, cholecalciferol or a vitamin D analog; and
   iii) a statin;
wherein the drug or combination of drugs of paragraphs a), b) and c) are administered to the patient by a delivery method selected from the group consisting of:
aa) localized delivery using: implanted or topically applied hydrogels, poloxamer gels, polysaccharide gels; nanomedicinal formulations; 3D printed gels; or microemulsions;
bb) 3D printed formulations;
cc) nanoscale drug delivery systems using liposomes and nanoparticles;
dd) a microneedle array; and
ee) transdermal delivery or implantable sponges soaked in drugs;
and wherein the dosage of the drug or combination of drugs is sufficient to make the drug or combination of drugs effective at inhibiting osteogenesis in mesenchymal stem cells within a selected anatomic distance from the site of delivery (e.g., within 0.1-12 cm; 0.1-6.0 cm; 0.1-4.5 cm; 0.1-2.0 cm; 0.1-1.0 cm; 0.1-0.5 cm; 0.5-1.5 cm) but which does not substantially inhibit osteogenesis in mesenchymal stem cells outside of the selected anatomic distance.

The method may be used to prevent or treat heterotopic ossification subsequent to spinal cord damage, traumatic injury, head or brain injuries, burns, bone fractures, muscle injuries, or joint replacement surgery. It may also be used to prevent or treat myositis ossificans; progressive osseous heteroplasia, fibrodysplasia ossificans progressiva or Albright's hereditary osteodystrophy. In the case of veterinary applications, the method may be used to prevent or treat myositis ossificans or fibrodysplasia ossificans progressiva in a cat or dog.

Hh pathway antagonists may be administered to patients at 0.5-500 mg/day; vitamin D, cholecalciferol or a vitamin D analogs may be administered at 100-3000 IU/day; and statins may be administered at 0.5-500 mg/day. In some embodiments one or more of these drugs may be encapsulated and/or in the form of nanoparticles.

Hh pathway antagonists may be ligands that bind to the Sonic receptor and prevent activation; an antibody that binds to either Sonic, Desert or Indian or to the receptor for these ligands; or an siRNA. Specific Hh pathway antagonists may be selected from the group consisting of: a) zerumbone epoxide; b) staurosporinone; c) 6-hydroxystauro-sporinone; d) arcyriaflavin C; e) 5,6-dihyroxyarcyriaflavin A; f) physalin F; g) physalin B; h) cyclopamine; i) HPI-1, HPI-2; HPI-3; or HPI-4; j) arsenic trioxide (ATO); k) sodium arsenite; l) phenylarsine; m) GANT-58; n) GANT-61; o) zerumbone; and p) inhibitors of the expression of the genes Ptch1, Gli1 or HIP. One preferred Hh pathway antagonist is arsenic trioxide (ATO) administered to said patient at a dosage of between 0.05 to 0.20 mg/kg/day.

Statins that may be used include Atorvastatin; Fluvastatin; Pravastatin; Rosuvastatin; Simvastatin; Pitavastatin; Cerivastatin; Lovastatin; and Mevastatin.

Preventing or Treating Heterotopic Ossification, Vascular Calcification, or Pathologic Calcification

In a second aspect, the invention is directed to a method for preventing or treating heterotopic ossification, vascular calcification, or pathologic calcification in a patient, comprising administering to the patient a drug selected from the group consisting of: a Hedgehog (Hh) pathway antagonist; vitamin D, cholecalciferol or a vitamin D analog; and a statin; or a combination of drugs selected from:
a) a combination of a Hedgehog (Hh) pathway antagonist together with:
   i) vitamin D, cholecalciferol or a vitamin D analog; or
   ii) a statin;
b) a combination of:
   i) vitamin D, cholecalciferol or a vitamin D analog; and
   ii) a statin; or
c) a combination of:
   i) an Hh pathway antagonist;
   ii) vitamin D, cholecalciferol or a vitamin D analog; and
   iii) a statin;
wherein the drug or combination of drugs of paragraphs a), b) and c) are administered to the patient by a delivery method selected from the group consisting of:
aa) localized delivery using: implanted or topically applied hydrogels, poloxamer gels, polysaccharide gels; nanomedicinal formulations; 3D printed gels; or microemulsions;
bb) 3D printed formulations or encapsulated drugs;
cc) nanoscale drug delivery systems using liposomes and nanoparticles;
dd) a microneedle array; and
ee) transdermal delivery or implantable sponges soaked in drugs;
and wherein the drug or combination of drugs is at a dosage sufficient to make the drug or combination of drugs effective at preventing or treating heterotopic ossification, vascular calcification, or pathologic calcification within a selected anatomic distance from the site of delivery (e.g., within 0.1-15 cm; 0.1-12 cm; 0.1-6.0 cm 0.1-6.0 cm; 0.1-4.5 cm; 0.1-2.0 cm; 0.1-1.0 cm; 0.1-0.5 cm; 0.5-1.5 cm) but which does not substantially inhibit bone formation or calcification outside of the selected anatomic distance.

The treatment methods described above may be used for patients (human or animal) with, or at risk of developing, ectopic bone formation or calcification of any origin, including spinal cord damage, traumatic injuries, head and brain injuries, burns, bone fractures, muscle injuries, and surgery. The methods should also be effective in patients with diseases or conditions that predispose them to ectopic bone formation or calcification, such as atherosclerosis or myocardial infarction, chronic inflammation, such as ankylosing spondylitis (a type of arthritis that mostly affects the lower part of the spine, and where it joins to the hips, known as the sacroiliac joints) and other complications of autoimmune conditions, perimyositis (inflammation of connective tissue around a muscle), and in genetic diseases such as osseous heteroplasia, fibrodysplasia ossificans progressiva, Albright's hereditary osteodystrophy and osteopetrosis. Veterinary uses include the treatment of myositis ossificans and fibrodysplasia ossificans progressiva in cats and dogs. In alternative embodiments, the amount or concentration of one or more released drugs at the furthest point of a given anatomic distance should be less than half of the amount or concentration at the nearest point. For example, the amount or concentration at 2.0 cm should be less that half of the amount or concentration at 0.1 cm.

Hh pathway antagonists may be administered to patients at 0.5-500 mg/day; vitamin D, cholecalciferol or a vitamin D analogs may be administered at 100-3000 IU/day; and statins may be administered at 0.5-500 mg/day. In some embodiments one or more of these drugs may be encapsulated and/or in the form of nanoparticles.

Hh pathway antagonists may be ligands that bind to the Sonic receptor and prevent activation; an antibody that binds to either Sonic, Desert or Indian or to the receptor for these ligands; or an siRNA. Specific Hh pathway antagonists may be selected from the group consisting of: a) zerumbone epoxide; b) staurosporinone; c) 6-hydroxystauro-sporinone; d) arcyriaflavin C; e) 5,6-dihyroxyarcyriaflavin A; f) physalin F; g) physalin B; h) cyclopamine; i) HPI-1, HPI-2; HPI-3; or HPI-4; j) arsenic trioxide (ATO); k) sodium arsenite; 1) phenylarsine; m) GANT-58; n) GANT-61; o) zerumbone; and p) inhibitors of the expression of the genes Ptch1, Gli1 or HIP. One preferred Hh pathway antagonist is arsenic trioxide (ATO) administered to said patient at a dosage of between 0.05 to 0.20 mg/kg/day.

Statins that may be used include Atorvastatin; Fluvastatin; Pravastatin; Rosuvastatin; Simvastatin; Pitavastatin; Cerivastatin; Lovastatin; and Mevastatin

Implantable Drug Delivery System

In a third aspect, the invention is directed to an implantable drug delivery system (also referred to herein as an implantable device) for directionally delivering two or more different drugs comprising:
a) a first section comprising a first therapeutic agent or combination of therapeutic agents wherein, upon implantation into a patient, said first section releases said first therapeutic agent or combination of therapeutic agents over a period of time;
b) a second section comprising a second therapeutic agent or combination of therapeutic agents wherein, upon implantation into a patient, said second section releases said second therapeutic agent or combination of agents over a period of time;
wherein said first section and said second section are separated by a barrier that inhibits or blocks the passage of therapeutic agents between the first and second sections and which limits the area in which the first and second therapeutic agents are released.

The first and second sections of the implantable drug delivery system may comprise polymeric gels (preferably hydrogels) that are separated by a barrier that is impermeable (preferably completely impermeable) to the first therapeutic agent or combination of therapeutic agents and is impermeable (preferably completely impermeable) to the second therapeutic agent or combination of therapeutic agents. The gels in the first and second sections may take the form of one or more polymeric gel layers with drugs or drug combinations interspersed or compartmentalized in the gel and with a barrier that is impermeable to drugs or combinations of drugs separating layers comprising the first therapeutic agent or combination of therapeutic agents from layers comprising the second therapeutic agent or combination of therapeutic agents. The implantable drug delivery systems can, optionally contain more than two sections and, in some embodiments the implantable drug delivery systems may be partially or completely made by 3-D printing. All of the different sections should be separated from one another by a barrier that is impermeable (preferably completely impermeable) to the drugs, that limits the area where drugs are released and that may, optionally, extend partly over and around sections to further limit where drugs are released. Each section of a device should deliver drugs or other agents directionally, i.e., the drugs and agents in a section should be at a higher concentration in the tissues, bone or other biological structures immediately adjacent to that section than in the tissues, bone or other biological structures immediately adjacent to the other sections of the device. In some embodiments one or more of these drugs may be encapsulated and/or in the form of nanoparticles.

The gels in the drug delivery system should be provided with an amount of drug such that, when the drug delivery system is implanted, each drug or combination of drugs is released at a rate such that the dosage of the therapeutic agent or combination of therapeutic agents is sufficient to be therapeutically effective only within a selected anatomic distance from the site of delivery (e.g., within 0.1-15 cm; 0.1-12 cm; 0.1-6.0 cm 0.1-6.0 cm; 0.1-4.5 cm; 0.1-2.0 cm; 0.1-1.0 cm; 0.1-0.5 cm; 0.5-1.5 cm). In an alternative embodiment, the amount or concentration of one or more released drugs at the furthest point of a given anatomic distance from the site of delivery should be less than half of the amount or concentration at the nearest point. For example, the amount or concentration at 2.0 cm from the site of delivery should be less that half of the amount or concentration at 0.1 cm from the site of delivery.

In a preferred embodiment, one section of the implantable drug delivery system comprises a drug or combination of drugs that promote bone formation and/or a section of the implantable drug delivery system comprises a drug or combination of drugs that prevent or treat heterotopic ossification, vascular calcification, or pathologic calcification. When present in a single device, drugs that promote bone formation and drugs that prevent or treat heterotopic ossification, vascular calcification, or pathologic calcification should be in separate sections that are separated by a barrier impermeable (preferably completely impermeable) to the drugs.

More generally drugs that may be present in the implantable drug delivery system may comprise: a chemotherapeutic drug, an analgesic, an antibiotic, an antibody, a hormone, an anti-inflammatory, an Hh pathway antagonist; vitamin D, cholecalciferol, a vitamin D analog, a statin, and/or an agent that promotes bone formation or combination of thereof. Generally, the drug or drugs in the first and second sections should have different therapeutic effects and, in some instances the drugs in one section will inhibit effects caused by the drugs in the other section. The sections of the implantable drug delivery system may include not only drugs but also other agents that may be of benefit to a patient and such agents may be used together with a drug or in the place of a drug. For example, the invention includes devices and methods of delivery in which one (or all) sections of a device contain saline or other agents to soothe or moisten tissues but do not have drugs for preventing or treating a specific disease or condition.

Orthopedic Uses of Drug Delivery Systems

In a preferred embodiment, the implantable drug delivery system comprises a first section that optionally has a first therapeutic agent or combination of therapeutic agents and a second section with a second therapeutic agent or combination of therapeutic agents, wherein the second therapeutic agent or combination of therapeutic agents prevent or treat heterotopic ossification, vascular calcification, or pathologic calcification. Preferably the second therapeutic agent is a drug selected from the group consisting of a Hedgehog (Hh) pathway antagonist; vitamin D, cholecalciferol or a vitamin D analog; and a statin; or a combination of drugs selected from:
a) a combination of a Hedgehog (Hh) pathway antagonist together with:
   i) vitamin D, cholecalciferol or a vitamin D analog; or
   ii) a statin; or
b) a combination of:
   i) vitamin D, cholecalciferol or a vitamin D analog; and
   ii) a statin; or
c) a combination of:
   i) an Hh pathway antagonist;
   ii) vitamin D, cholecalciferol or a vitamin D analog; and
   iii) a statin.

In a preferred embodiment, the first therapeutic agent is present and preferably comprises a therapeutic agent or combination of therapeutic agents that promote bone formation or growth. These therapeutic agents may comprise bone morphogenetic protein 2 (BMP-2) or bone morphogenetic protein 7 (BMP-7).

When the drug delivery system is implanted, each therapeutic agent or combination of therapeutic agents is released at a rate such that the dosage of the therapeutic agent or combination of therapeutic agents is sufficient to be therapeutically effective only within a selected anatomic distance from the site of delivery (e.g., within 0.1-15 cm, 0.1-12, cm; 0.1-6.0 cm; 0.1-4.5 cm; 0.1-2.0 cm; 0.1-1.0 cm; 0.1-0.5 cm; 0.5-1.5 cm) and where there is not a substantial therapeutic effect outside of the selected anatomic distance. In an alternative embodiment, the amount or concentration of one or more released drugs at the furthest point of a given anatomic distance should be less than half of the amount or concentration at the nearest point. For example, the amount or concentration at 2.0 cm should be less that half of the amount or concentration at 0.1 cm.

Therapeutic agents that may be present in the second section of the implantable drug delivery system include one or more of: a ligand that binds to the Sonic receptor and prevents activation; an antibody that binds to either Sonic, Desert or Indian or to the receptor for these ligands; or an siRNA. Specific drugs include one or more of: a) zerumbone epoxide; b) staurosporinone; c) 6-hydroxystauro-sporinone; d) arcyriaflavin C; e) 5,6-dihyroxyarcyria-flavin A; f) physalin F; g) physalin B; h) cyclopamine; i) HPI-1, HPI-2; HPI-3; or HPI-4; j) arsenic trioxide (ATO); k) sodium arsenite; 1) phenylarsine; m) GANT-58; n) GANT-61; o) zerumbone; and p) inhibitors of the expression of the genes Ptch1, Gli1 or HIP. A preferred drug is arsenic trioxide (ATO). Preferred statins are Atorvastatin; Fluvastatin; Pravastatin; Rosuvastatin; Simvastatin; Pitavastatin; Cerivastatin; Lovastatin; and Mevastatin.

Methods of Using Drug Delivery Systems Therapeutically

The present invention also encompasses methods of preventing or treating a disease or condition in a patient by the localized delivery of a drug or agent in which any of the implantable drug delivery systems described herein are implanted and then directionally release a therapeutically effective amount of drug only within a selected anatomic distance from the site of delivery. For example, the implantable drug delivery systems may be implanted in a patient with a solid tumor and oriented so that an antitumor or antimetastatic agent is released from a first section at, or adjacent, to the site of tumor growth. These agents may be released in such a way that a therapeutically effective amount of drug is restricted to a distance of 0.1-10.0 cm; 0.1-6 cm; 0.1-4.5 cm; 0.1-2.0 cm; 0.1-1.0 cm; 0.1-0.5 cm; etc. from the site of delivery. A second drug may also be released from a different section of the implantable drug delivery system at a site different from the site of tumor growth. This may be a drug that helps prevent the spread of tumor cells or an agent that offsets detrimental effects caused by the release of the antitumor or antimetastatic agent from the first section of the implantable drug delivery system.

Alternatively, treatment methods may be directed to a patient that has, or is at risk of developing heterotopic ossification, vascular calcification, or pathologic calcification. Particular patients that may be treated include patients with one or more broken bones or that have undergone orthopedic surgery such as hip replacement surgery or surgery on the spinal column and patients with atherosclerosis, chronic inflammation, complications due to of autoimmune conditions, perimyositis, osseous heteroplasia, fibrodysplasia ossificans progressiva, Albright's hereditary osteodystrophy, osteopetrosis or that have had a myocardial infarction. As discussed elsewhere herein, drugs or drug combinations that are known to be effective for such conditions, including Hh pathway antagonists, vitamin D, cholecalciferol or a vitamin D analog, statins and combinations of these drugs, may be used and implantable drug delivery systems may be oriented to release these drugs at sites where heterotopic ossification, vascular calcification, or pathologic calcification has occurred or is believed to be likely to occur. Other agents such as saline, buffer, analgesics or antibiotics may also be released. In cases where there has been bone injury due to an accident or due to surgery, one section of the devices may be positioned so that it is adjacent to and releases drugs in the direction of, the site of injury. These drugs may promote bone growth and include BMP-2 and BMP-7. Another section of the implantable drug delivery system may release drugs for preventing or treating heterotopic ossification, vascular calcification, or pathologic calcification in a direction away from the site of injury and into surrounding tissue.

In a preferred embodiment, the invention incudes a method of treating a patient that has undergone orthopedic surgery resulting in an anatomic treatment site where bone or cartilage formation is needed, or has undergone trauma resulting in at least one anatomic treatment site comprising a broken bone or damaged cartilage. In these cases, the method comprises implanting in the patient the implantable drug delivery systems described herein wherein the implantable drug delivery system is positioned adjacent to the anatomic treatment site (i.e., the site that has been treated surgically or where traumatic injury has occurred) and is oriented so that a first section comprising a first therapeutic agent or combination of therapeutic agents is closest to the anatomic treatment site. This section releases therapeutic agents (e.g., BMP-2 and BMP-7) that promote bone formation, bone growth or cartilage repair at that site.

A second therapeutic agent or combination of therapeutic agents is released from a separate, second section of the implantable drug delivery system. This second drug or combination of drugs may comprise a Hedgehog pathway antagonist, vitamin D, cholecalciferol, a vitamin D analog, or a stain; or a combination comprising:
a) a combination of a Hedgehog (Hh) pathway antagonist together with:
   i) vitamin D, cholecalciferol or a vitamin D analog; or
   ii) a statin;
b) a combination of:
   i) vitamin D, cholecalciferol or a vitamin D analog; and
   ii) a statin; or
c) a combination of:
   i) an Hh pathway antagonist;
   ii) vitamin D, cholecalciferol or a vitamin D analog; and
   iii) a statin.

Specific therapeutic agents that may be used as a second therapeutic agent may be selected from the group consisting of: a ligand that binds to the Sonic receptor and prevents activation; an antibody that binds to either Sonic, Desert or Indian or to the receptor for these ligands; or an siRNA. Alternatively, it may be selected from the group consisting of: a) zerumbone epoxide; b) staurosporinone; c) 6-hydroxystauro-sporinone; d) arcyriaflavin C; e) 5,6-dihyroxyarcyriaflavin A; f) physalin F; g) physalin B; h) cyclopamine; i) HPI-1, HPI-2; HPI-3; or HPI-4; j) arsenic trioxide (ATO); k) sodium arsenite; 1) phenylarsine; m) GANT-58; n) GANT-61; o) zerumbone; and p) inhibitors of the expression of the genes Ptch1, Gli1 or HIP. Statins that may be used include Atorvastatin; Fluvastatin; Pravastatin; Rosuvastatin; Simvastatin; Pitavastatin; Cerivastatin; Lovastatin; and Mevastatin.

Drug Forms

It will be understood that therapeutic agents that act in treating or preventing a disease or condition in the treatment methods and implantable drug delivery systems described herein may be used in any pharmaceutically acceptable form, i.e., any form which maintains therapeutic activity and which does not cause unacceptable adverse effects when administered. For example, a compound may be in the form of a pharmaceutically acceptable salt, ester or pro-drug. Therapeutic agents may optionally be encapsulated and may take the from of nanoparticles. In some embodiments, sponges or gels may be implanted that have been soaked in drugs or in which drugs have been incorporated by some other means.

### BRIEF DESCRIPTION OF THE DRAWINGS

FIG. 1 illustrates one possible arrangement of an implantable gel. In this example, one or more drugs that promote bone growth (Drug A) are delivered in the direction of damaged bone and one or more drugs preventing ectopic bone formation (Drug B) are released in a direction toward tissue and away from the bone. The striped portions of the figure represent a matrix, e.g., a hydrogel or polymer. Drugs may be interspersed in the matrix and released over a period of time. Release may be due, for example, to the diffusion of a drug out of the matrix, due to the dissolution of the matrix itself or due to some other release mechanism. “A” and “B” may each separately represent a single drug or a combination of drugs. In addition to drugs for specific diseases or conditions, other prophylactic agents, excipients, carriers, etc., may also be present. For example, analgesics or antibiotics may be included.

Between the drug matrices containing Drug A and Drug B is a section that is impermeable to the drugs (gray portion of the figure) and that also extends along the sides of the implantable device. This may be a polymer, gel or other material. In addition to being used for broken bone and traumatized tissue, the implant may be used for other treatments and employ other drugs. For example, the implant might be used after orthopedic surgery to deliver drugs to a surgical site and other drugs to the surrounding tissue.

FIG. 2 illustrates a multilayered implantable hydrogel device. In this design, there is a topmost gel layer that has a first drug or combination of drugs (e.g., in the form of nanoparticles). These may be interspersed through all or a portion of the gel as shown in the topmost and bottommost layers. In the intermediate top layer and intermediate bottom layer the drug particles are still interspersed in a gel layer but the particles have been confined to a oblong section in the layer. Alternatively the oblong section may represent a compartment within the gel layer from which drugs diffuse. The compartment may optionally take the form of a well that may or may not have the same consistency as the rest of the layer. A layer may have one or more such compartments and they may be arranged in any manner in the layer. When the device is implanted, drugs will diffuse out of the gel and into the nearest biological matter.

The topmost gel layer is positioned over a first intermediate layer which contains a second drug or combination of drugs that may, or may not, be the same as those in the topmost layer. The composition of the intermediate gel layer may be altered from that of the topmost layer to change the rate of diffusion of interspersed drugs. For example the density of the hydrogel might be different or there may be pores of a different average diameter. The gels in the different layers may be the same or different and a gel may be homogeneous throughout a layer or there may be areas where the gel composition or density is different. In the figure, the different markings on top and bottom layers compared to the intermediate layers are intended to represent gels of different consistency. The topmost and bottommost layers are homogeneous whereas the intermediate layers are not. Optionally, additional intermediate layers may also be present.

The top layers lie over a blocking layer that is impermeable, preferably completely impermeable, to drug. This may be a polymer or some other biologically acceptable substance. On the opposite side of the blocking layer there may be one or more intermediate bottom layers each with a drug or combination of drugs. In the figure, there is a single intermediate bottom layer with a third drug or drug combination dispersed or compartmentalized in its structure (i.e., present in one or more compartments from which drugs diffuse). Finally, there is a bottommost layer that has a fourth drug or drug combination that is the same or different from that of the intermediate bottom layer. The composition of drugs in the bottom layers of the device should generally be different than the composition in the top layers and, when implanted, the device should release the drugs in the top layers in one direction and the drugs in the bottom layers in a different direction. By way of example, the device might be implanted so that the topmost layer is adjacent to a site of bone fracture and releases drugs that promote bone regrowth. The bottommost layer would face away from the bone and release drugs that inhibit bone formation into adjacent tissue. The distance between the top layers and the bottom layers can be altered by changing the thickness of the blocking layer.

FIG. 3 illustrates the steps that might be used in the surgical repair of a broken hip or in other hip surgery and depicts, inter alia, the anatomy surrounding the acetabulum and the implantation of a device. In the example, a surgeon would retract the tissue at the site of fracture and surgically treat the site. He would then implant a hydrogel device which has been soaked, or otherwise provided, with “NP-101” (a combination of a hedgehog pathway antagonist, vitamin D, cholecalciferol or a vitamin D analog and a statin). The NP-101 hydrogel layer lies on top of a barrier layer impermeable to NP-101 and the device is implanted so that NP-101 containing layer faces away from the bone and toward the retracted tissue. The impermeable layer is positioned between the retracted tissues and the injured bone and may, optionally, have one or more additional hydrogel layers on the side opposite from the NP-101 containing layer and facing toward the bone. The additional layer or layers may have been provided with a solution containing drugs or other agents that promote bone repair and release these drugs or other agents at the site of injury. The layers facing the bone and those facing the tissue may also contain other drugs or deliverable components, such as antibiotics or analgesics. After the device has been implanted, the retracted tissue would be rejoined and the hip would be allowed to heal.

### DETAILED DESCRIPTION OF THE INVENTION

### A. Definitions

Co-timely Administration: The approaches to drug delivery described herein may release different therapeutic compounds at about the same rate. However, there may be instances where one drug is released much faster than another or where a formulation releases drugs at different times. It would also be possible to use one gel or device to release one compound and one or more other devices to release other compounds in a given combination. In such cases, the compounds should preferably be administered by the gels or devices in a co-timely manner. By way of example, “co-timely administration” means administration of a subsequent drug to prevent or treat heterotopic ossification, vascular calcification, or pathologic calcification during the time when a previously administered, first drug, is still present in an amount in a patient that is therapeutically effective in combination with the second drug. Thus, drugs must be given in close enough temporal proximity that they can have a cumulative effect. If a third drug is given, then this should be done while both the first and second drugs are present in therapeutically effective amounts when in combination with the third.

Concomitant Administration: As used herein, the term “concomitant administration” means that drugs are administered within one hour of one another.

Heterotopic Ossification: Heterotopic ossification is the deposition of bone at sites in the body where it does not belong. Unless otherwise indicated, the term as used herein refers to bone formation at a site that is abnormal and undesired wherever that site happens to be and regardless of the cause.

Vascular calcification: Vascular calcification refers to the deposition of calcium in blood vessel structures and is often associated with atherosclerosis. (Bostrom, et al., J. Clin. Invest. 91:1800-09 (1993)). The consequences of calcification of blood vessels can be severe and may lead to congestive heart failure, aortic stenosis and weakened vasomotor responses.

Drug or therapeutic agent: As used herein the terms “drug” and “therapeutic agent” are used interchangeably and refer to any agent that causes an improvement in a clinically recognized characteristic or symptom associated with a disease or condition. The drug or therapeutic agent may be a natural product, a biologic, a chemical compound, a dietary supplement, a nutriceutical, or any other substance.

Pathologic Calcification: For our purposes herein, pathologic calcification may be considered to be the deposition of calcium salts in soft tissue causing a hardening, but not bone formation. The term includes vascular calcification but also includes calcification outside of the vasculature.

Differences in Combinations of therapeutic agents: Unless indicated either expressly or by context, when reference is made herein to one combination of therapeutic agents differing from a second combination, this refers to a difference in one or more of the specific drugs present, as opposed, for example, to simply a change in concentration.

Vitamin D, Cholecalciferol and Vitamin D Analogs: As used herein “cholecalciferol” refers specifically to vitamin D3 whereas the term “vitamin D” comprises all forms of vitamin D (including vitamin D2 (ergocalciferol) and D3 (cholecalciferol)) and combinations of these forms. Unless otherwise indicated, dosages or quantities recited refer to the total combined amount of all forms of vitamin D administered to a patient or present in a composition. The term “vitamin D analog refers to any compound (other than a naturally occurring form of vitamin D) which has vitamin D biological activity and especially any such compound that binds to, and activates the vitamin D receptor (i.e., the calcitrol receptor). Such receptors may be found, for example in human osteoblasts, hepatocytes or immune cells. Examples of vitamin D analogs include but are not limited to those in the following US patent references (all of which are incorporated by reference herein in their entirety): (U.S. Pat. Nos. 7,985,744; 8,198,263; 7,659,421; 7,211,680; 7,115,758; 7,112,579; 7,074,777; 6,538,145; 6,359,152; 6,277,837; 6,124,276; 6,043,385; 6,013,814; 5,945,410; 5,756,733; 5,700,791; 5,665,716; 5,446,035; 5,232,836; 4,891,364; 4,857,518 4,851,400).

Statins: Statins are recognized in the art as a distinct drug class that act as inhibitors of 3-hydroxy-3-methylglutaryl coenzyme A reductase (HMG-CoA reductase), an enzyme involved in the synthesis of cholesterol. Structurally, they are characterized by a dihydroxyheptanoic acid group (sometimes in the form of a lactone) which forms a structure resembling HMG-CoA (the substrate of HMG-CoA reductase). This group is attached to a variety of ring systems (including aromatic, heterocyclic or aromatic-heterocyclic, unsubstituted or substituted, mono-, di- or poly-cyclic ring systems). Specific examples of statins include Atorvastatin; Fluvastatin; Pravastatin; Rosuvastatin; Simvastatin; Pitavastatin; Cerivastatin; Lovastatin; and Mevastatin. These specific compounds and all compounds recognized in the art as being a member of the statin drug class are included within the scope of the invention.

Hedgehog pathway antagonists: The hedgehog signaling pathway is involved in the differentiation of cells during embryonic development and also appears to play a role regulating adult stem cells. Inhibition of this pathway has been reported to decrease the proliferation and clonogenicity of human mesenchymal stem cells which are known to be capable of differentiating into, inter alia, osteoblasts (see Plaisant, et al., PLoS One: 6(2):e16798 (2011)). Any inhibitor of this pathway identified in the art as being effective is within the scope of the invention regardless of its mechanism of action. This includes: small molecules that block the binding of a hedgehog ligand (Desert, Indian or Sonic) to its receptor; antibodies that target either ligand or receptor; agents that block intracellular activation after receptor binding; and agents that block gene expression, such as siRNAs. Examples of specific inhibitors include: a) zerumbone epoxide; b) staurosporinone; c) 6-hydroxystaurosporinone; d) arcyriaflavin C; e) 5,6-dihyroxyarcyriaflavin A; f) physalin F; g) physalin B; h) cyclopamine; i) HPI-1, HPI-2; HPI-3; or HPI-4; j) arsenic trioxide (ATO); k) sodium arsenite; 1) phenylarsine; m) GANT-58; n) GANT-61; o) zerumbone; and p) inhibitors of the expression of the genes Ptch1, Gli1 or HIP. Examples of other Hh pathway antagonists that may be used in the invention include, but are not limited, to those in the following US patent references (all of which are incorporated by reference herein in their entirety): U.S. Pat. Nos. 9,427,431; 9,409,871; 9,346,791; 9,345,699; 9,321,761; 9,278,961; 9,216,964; 9,174,949; 9,173,869; 9,149,527; 9,096,686; 9,073,835; 9,000,023; 8,835,648; 8,802,639; 8,778,927; 8,759,367; 8,530,456; 8,486,400; 8,410,601; 8,273,747; 8,101,610; 8,030,454; 7,741,298; 7,407,967; 6,683,108; and 6,291,516.

Therapeutically effective amount: The term “therapeutically effective amount” means a dosage of drug that provides the specific pharmacological response for which the drug is administered in a significant number of subjects in need of such treatment. For example, this might be a sufficient dose of each active drug administered which, in combination, reduces the number of patients developing a disease or condition such as heterotopic ossification or pathologic calcification (e.g., after arthroscopic surgery, spinal injury, trauma, head or brain injuries, bone fractures or burns) by at least 15% (preferably at least 30% and more preferably at least 50%) relative to clinically matched patients that are not treated. In patients that are diagnosed as having a disease or condition (e.g., heterotopic ossification), a therapeutically effective amount is a dosage sufficient to reduce the clinical symptoms associated with the disease or condition to a greater degree in at least 15% of patients receiving treatment (preferably at least 30% and more preferably at least 50%) over a period of 3 to 12 months relative to clinically matched patients that are not treated. By way of example, clinical symptom improvement may take the form of a greater reduction in pain, swelling, or pressure or a greater shrinkage of bone or calcium deposits. Note that reference to a “specific pharmacological response for which the drug is administered in a significant number of subjects in need of such treatment” is a recognition that a “therapeutically effective amount,” administered to a particular subject may not be effective in that patient even though such dosage is deemed to be therapeutically effective by those skilled in the art.

Unit dosage form: The term “unit dosage form” is defined as a single drug administration entity. It applies to drugs delivered transdermally in a single patch or as part of a specified amount of gel, foam, ointment or other device that is implanted or applied topically to a patient. These devices or gels will then release therapeutic compounds over a period of time.

Within a selected anatomic distance: A primary objective of the drug delivery approaches described herein is to localize compounds that have a therapeutic effect to the area where they are needed and to minimize the extent to which the drugs have effects elsewhere. For example, the release of drugs that promote bone repair may occur at or near a site of fracture and be at a therapeutically effective concentration there but at a lower concentration that is not therapeutically effective a short distance (e.g., one centimeter) away from the bone. Drugs inhibiting bone formation may be released at or near tissues but be present at too low of a concentration at the site of fracture to affect bone regrowth. The exact distance selected will vary depending on the circumstances presented by a specific patient and the disease or condition for which the drug is administered. For example, to prevent or treat abnormal bone formation in tissues adjacent to a fracture, the selected distance from a drug delivery site might be very short (perhaps only a few millimeters, e.g., 1-9 mm) whereas the selected distance may be much longer (e.g., 1-15 cm) when treating traumatized tissue at a site more distant from a fracture. In all cases, the distance selected for delivery of a therapeutically effective amount of drug will be determined by the amount of drug loaded in a gel or device and the rate at which that drug is released. Specific information on release rates can be determined empirically using methods that are standard in the art.

Site of Delivery: Unless otherwise indicated either expressly or by context the term “site of delivery” or “drug delivery site” as used in herein, and in the context of an anatomic distance in which a drug is therapeutically effective, refers to the site where a drug or agent passes out of, or is otherwise released from, a device and into the surrounding anatomic structures. Thus, the site of delivery will be different for different sections of a device and for different areas of a single section.

Patient: As used herein, the term “patient” refers to any human (or in the case of veterinary medicine, any animal) that is being administered one or more drugs to treat or prevent a disease or condition, or an abnormality resulting from a disease or condition (e.g., abnormal bone formation).

Substantially inhibit: As used herein, a therapeutic agent or combination of therapeutic agents substantially inhibits a process when the agent or combination of agents reduces the rate at which the process proceeds by more than 20% (preferably by more than 50%, more preferably by more than 75% and still more preferably, by more than 90%) compared to the rate in the absence of the agent or combination of agents. In the context of inhibiting an abnormal process such as heterotopic ossification, it is preferable that the rate of the ossification process be inhibited by more than 50%, more preferably, by more than 75%, and still more preferably, by more than 90%. Conversely, a therapeutic agent or combination of therapeutic agents does not substantially inhibit a process when it does not reduce the rate at which it occurs by more than 20% compared to the rate in the absence of the agent or agents. In the context of inhibiting a normal process such as bone healing after a fracture, it is preferable that the rate of the process not be reduced by more than 15% and more preferably by not more than 10%.

Substantial therapeutic effect: As used herein, a therapeutic agent or combination of therapeutic agents has a substantial therapeutic effect when a clinically recognized benefit of the treatment is realized in a significant portion of the patients treated (e.g., at least 10%, 20%, 30% or 50%). For example a chemotherapeutic agent would have a substantial therapeutic effect if it caused a statistically significant reduction in tumor size in a significant portion of the patients treated (e.g., at least 10%, 20%, 30% or 50%). If the amount of drug is below the level producing a clinically recognized benefit of the treatment in a significant portion of the patients treated, then it is not considered to have a substantial therapeutic effect.

Effective at inhibiting: As used herein, term “effective at inhibiting” means that the presence of one or more therapeutic agents reduces the rate at which a process (e.g., bone formation in response to a fracture) proceeds by more than 20% and preferably by more that 50%, 75% or 90%).

Diffusion: In general, the term “diffusion” refers to the net passive movement of drug from a region in which it is in relatively high concentration to regions of lower concentration. When referring to the release of drugs from gels or hydrogels as used herein, it will be recognized that diffusion also encompasses release aided by the dissolution of an implanted gel.

Drug delivery system/device: Unless indicated either expressly or by context, these terms as used herein are interchangeable.

### B. Advantages of Implantable Gel-Based Drug Delivery Systems

Current treatment methodologies for many orthopedic problems involving the systemic, untargeted administration of medications may result in unintended side effects, implant failure and lack of intended efficacy. As a result, patients may require invasive surgery (or a second surgery) to correct or undo unwanted consequences of a treatment. It therefore is desirable to provide formulations and methods that limit the delivery of medications to the tissues needing treatment. For example, a delivery method would, ideally, separate and compartmentalize the activity of medications promoting bone healing or other desired effects, such as cartilage formation, and medications preventing pathologic bone formation in traumatized tissue.

In this regard, the polymeric, preferably hydrogel, based delivery systems discussed herein have the advantage of being able to deliver a first drug in a first direction and at least a second drug in a different direction. This can be accomplished by having one section or area of the gel impregnated with the first drug, and a distinct, preferably opposite, section (or area) impregnated with a second drug. In order to restrict the direction in which drugs can go, there is a barrier (preferably a polymeric barrier) that separates and segregates these sections or areas and the drugs that they contain. By preventing passage of drugs, release of the first drug from the polymer can be confined to one direction and release of the second drug to a different, preferably essentially opposite, direction.

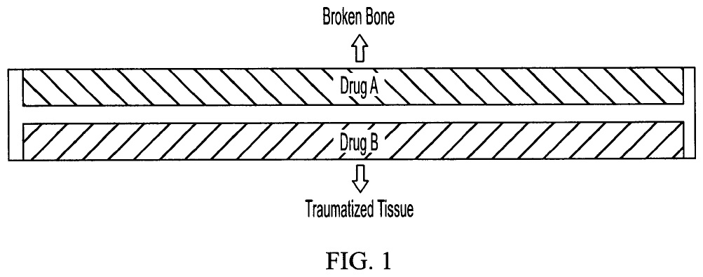

The advantage of this can be seen in FIG. 1 which illustrates a situation in which a patient has a broken bone (e.g., due to an injury or surgery) and tissue around the bone which has, or is susceptible to, pathologic ossification or calcification (e.g., due to trauma). An implantable delivery system may be prepared in which there are different sections of hydrogel. A first section contains Drug A that is a factor (or group of factors) that promote bone growth. For example, this section might contain bone morphogenetic proteins that induce the formation of bone and cartilage (see Lo, et al., Adv. Drug Delivery Rev. 64(12):1277-1291 (2012); Wang, et al., Exp. Ther. Med. 15(5):4298-4308 (2018); Mumcuoglu, et al., J. Translational Sci. 3(5):1-11 (2017), each of which is hereby incorporated by reference herein in their entirety). Two of these proteins BMP-2 and BMP-7 have been approved by the FDA for some clinical uses.

The drug delivery system is implanted adjacent to, or in close proximity to, the site needing treatment with Drug A (a broken bone in the figure) and with the gel section containing Drug A closest to and facing this site. The hydrogel then releases Drug A in the direction where treatment is needed (i.e., in the direction of the broken bone, the “Drug A treatment site”). In order to prevent Drug A from being released elsewhere, the first section is surrounded by a barrier in the form of a polymer or other substance that does permit the passage of the drug (shown in gray in FIG. 1).

A second section of the drug delivery system contains Drug B which, in the present example, might be one of the drugs or drug combinations described herein as preventing or treating heterotopic ossification, vascular calcification, or pathologic calcification, e.g., a combination of an Hh pathway antagonist; vitamin D, cholecalciferol or a vitamin D analog; and a statin (see also US 2018/0071319). This section of the implant is separated from the first section by the barrier (which also does not permit the passage of Drug B), and faces away from the Drug A treatment site and toward the area where treatment with Drug B is needed, i.e., the “Drug B treatment site.” Like Drug A, the release of Drug B in any direction except in the direction of damaged tissue (the Drug B treatment site) is blocked by the barrier (or a second barrier). Thus, each drug is concentrated at the site where it can be most effectively used and inhibited from going to sites where it may cause an adverse effect.

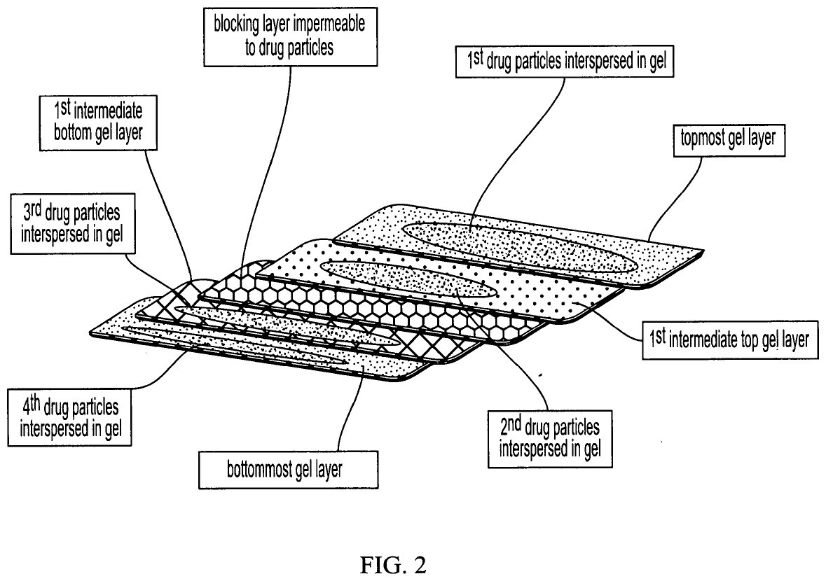

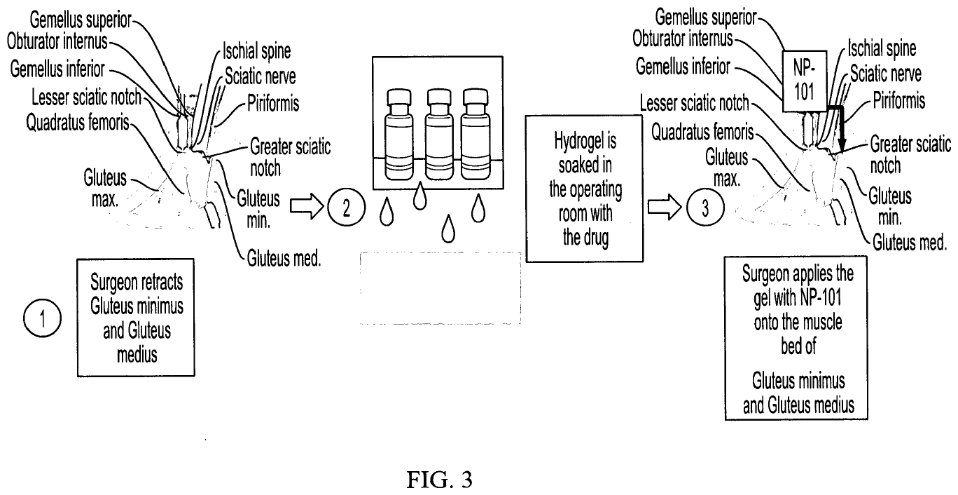

An alternative design may have multiple hydrogel layers as shown in FIG. 2. The impermeable barrier layer as shown may, if desired, be extended to further limit the direction of drug release. For example it may be designed to cover the side edges of top or bottom polymeric layers as in FIG. 1. The steps for implantation of a device for hip repair are illustrated in FIG. 3. Similar steps could be used for other sites of trauma or injury.

The implantable drug delivery systems can be used wherever drug segregation of the type described above is desired and, in addition to drugs for the treatment or prevention of diseases or conditions, other agents (e.g., saline, prophylactic drugs, analgesics, antibiotics, anti-inflammatories, drugs for pain management etc.) can be delivered either concurrently or alone. The system can be used post-surgically near the spine or elsewhere, or for treating other types of trauma that an individual might receive.

It should be apparent that the barrier and drug delivery sections of devices can be arranged in many different ways and that use is not restricted to the situation where there is damaged bone but extends to other clinical problems and other drugs. In this regard, all of the procedures discussed herein could also be used in cases where cartilage formation is needed, e.g., in elbow or knee joints after trauma or surgery or, more generally, in situations where it is desirable to locally release one therapeutic at a treatment site and a second therapeutic or prophylactic agent at a different site and to keep the first and second drugs separate from one another.

It should also be recognized that there are instances when just having a gel with a barrier that prevents the delivery of a drug to areas where it is not needed or desired would be advantageous. In the example discussed above, confining delivery of Drug A to the Drug A treatment site concentrates the drug where it is needed and could thereby lower the amount of drug that must be delivered for a therapeutic effect. Similarly, confining Drug B to the Drug B treatment site may avoid unwanted effects at other sites. By way of example, even if a drug promoting bone formation were given systemically to a patient, having a gel that blocked delivery of Drug B to the Drug A treatment site would be advantageous.

Release of a therapeutic or prophylactic agent is generally passively controlled by diffusion. Release rates can be adjusted by altering the geometry of the gel, the choice of polymer and the concentration of drug relative to the spatial positioning within the gel. Release may also be aided by, or caused by, the dissolution of the gel. Thus, altering the rate at which a gel dissolves provides an additional means for controlling the rate of drug delivery.

The quantity of therapeutic or prophylactic agent provided for release from one portion of an implant need not be the same for other areas and the time of release of the drugs may differ. The partition serving as a barrier may be created from an especially thick or dense polymer or by any other material compatible with use in the delivery system, that is acceptable for use therapeutically and that blocks the passage of drugs. Local delivery of a therapeutic or prophylactic agent may take place at joints or spinal discs, muscle or cartilage, ligaments or another soft tissue type.

### C. Characteristics of Gel Based and Polymeric Systems

Hydrogels

A preferred method of delivery in the methods described herein is using implanted or topically applied gels, especially hydrogels, incorporating natural polymers, especially polysaccharides like chitosan, alginate, gelatin etc. Hydrogels are characterized by nontoxicity, biocompatibility, biodegradability and abundant availability. They may also provide a cushioning effect to injured soft tissue. A hydrogel impregnated with agents that treat a disease or condition (e.g., heterotopic ossification, vascular calcification, and pathologic calcification) allows the precise release of the active ingredients to the site where treatment is needed (e.g., a site of suspected or diagnosed soft tissue ossification).

The hydrogel delivery vehicle will be loaded with, for example a drug or drug combination described herein and gradually release therapeutic compounds as the gel material breaks down after placement or injection at the site of the soft tissue injury. The hydrogel may take the form of an injectable sponge made up primarily of chitosan, alginate, and gelatin (see Bencherif, et al., Proc. Nat'l Acad. Sci. 2012; DOI: 10.1073/pnas.1211516109). This sponge-like gel is formed through a freezing process called cryogelation. The drug-loaded gel can be placed at or near a site via guided injection; e.g., using a titanium rod to deliver the gel formulation to injured or diseased soft tissues. The gel can be administered through the shaft of the rod, via incision and delivered in the form of a “thermoresponsive” pluronic formulation or “pH-responsive” polycarbophil formulation. The gel can also be administered as a combination of both.

Poloxamer Gels

Poloxamers are a family of ABA block copolymers, in which a hydrophobic poly (propylene oxide) (PPO) block is sandwiched between two hydrophilic poly (ethylene oxide) (PEO) blocks. The general structural formula of a poloxamer is ExPyEx, where x and y denote the number of ethylene oxide and propylene oxide monomers per block. In general, poloxamers behave like nonionic surfactants due to the nature of their block units. In certain solvents like water, these polymers form various structures, ranging from micellar to gel-like features, depending upon the length of the polymer subunits, concentration, and the temperature. The relatively dehydrated core of the micelle facilitates the incorporation of hydrophobic drugs in aqueous poloxamer solutions. Concentrated aqueous solutions of poloxamers form gels above the CGC (critical gelation concentration), when the temperature is raised above the CGT (critical gelation concentration). The gelation is due to physical entanglement and packing of the micellar structures. This is a reversible process and below the CGT these gels remain fluid, a behavior which can be advantageously exploited for administration to the smaller orifices. Concentrated micellar solutions of poloxamers can transform from being a mobile fluid at room temperature to immobile gel at body temperature. This reversible process may be exploited for use as an in situ gelling drug delivery system.

Poloxamers have been studied for drug and gene delivery applications due to their biocompatibility and low toxicity. Poloxamer 407 (P407) is an important member with a nominal weight of 12600 Da, and 70% of a hydrophilic block. P407 has been approved as a formulation adjuvant in oral solutions, ophthalmic solutions, periodontal gels and topical emulsions. P407 gels have also been evaluated for ocular periodontal, dermal vaginal, subcutaneous, nasal, rectal and intratumoral delivery.

Compared to chemically cross-linked gels, poloxamer gels are easily diluted by body fluids. However, ionotropic gelation of mucoadhesive polymers in P407 gels may be used to overcome this fast dissolution. The use of a double syringe system facilitates drug loading and handling during administration and in situ ionotropic gelation of chitosan may be used to restrict dissolution and release of drug. The dual syringe approach may also be adapted for the in situ formation of nanoparticles dispersed in P407 gels loaded with, e.g., a drug or drug combination described herein.

Controlled release formulations based on thermoreversible poloxamer gels are suitable for the delivery of the drug combinations described herein. Co-solvents (DMSO, ethanol), mucoadhesive polymers (chitosan, alginate) and salts (sodium tripolyphosphate, CaCl2) may be used to enhance the applications of poloxamer 407 (P407) formulations. The impact of these additives on the micellization and gelation properties of P407 may reduce release rates of compounds and increase their plasma half-life. Since the disclosed combinations may include both hydrophobic and hydrophilic components, both behaviors have to be taken into account when using a membrane/membrane-free experimental setup.

Polysaccharide Gels

Polysaccharides are polymers of monosaccharides, which have a large number of reactive groups. Chitosan is a positively charged polysaccharide, while alginate and pectins represent negatively charged polysaccharides. As a natural biomaterial, these are highly stable, safe, non-toxic, hydrophilic and biodegradable. Gels based on such polyelectrolytes may be cross-linked using ionic interactions and are called ionotropic gels. Alginate and pectin form ionotropic gels with multivalent cations such as Ca²⁺, whereas chitosan may be crosslinked with poly-anions such as sodium tripolyphosphate (TPP).

Gels formed from the crosslinking of polysaccharides or polysaccharide-protein conjugates, e.g., using chitosan, hyaluronic acid, alginate or dextran and either gelatin or collagen (see e.g., U.S. Pat. No. 7,138,373 or 8,877,243, each hereby incorporated by reference in their entirety) may be used in delivering compounds. The most preferred gels are alginate-based polysaccharide hydrogels which may be made and used according to methods well known in the art (see e.g., Jain, et al., “Alginate drug delivery systems: application in context of pharmaceutical and biomedical research,” Drug Development and Industrial Pharmacy 40:1576-1584 (2014); Jana, et al., “Alginate Based Nanocarriers for Drug Delivery Applications,” Curr. Pharm Design 22:3399-410 (2016); Tonnesen, et al., “Alginate in Drug Delivery Systems,” Drug Development and Industrial Pharmacy 28(6):621-630 (2002), each of which is hereby incorporated by reference in its entirety).

3-D Printed Gels

3-D printing technology allows surgeons to select from a variety of shapes and materials to fit the physiology of the patient. For example, an acetabular fracture will require a different biomaterial deposition compared to an elbow fracture. A 3-D approach will allow a customized gradient of the active pharmaceutical ingredient. For example, a drug or drug combination described herein may be concentrated at one end of the gel, while the other part of the gel has excipients only, effectively serving as a buffer.

This approach has the potential for producing medicines which allow patients to be given an accurate and personalized treatment regime. The active agents used in the present combinations can be used either as a single blend, or potentially as layers in a multi-layer printed gel. For example, patients could have their treatment and dosage determined using identified HO biomarkers. Their individual, personalized medicines could potentially then be manufactured at the point of care. 3D printed delivery systems especially systems utilizing hydrogels may be used for other drugs as well.

### D. Alternatives to Implantable Gels

As an alternative to gels and polymers, the delivery methods and devices described below can potentially be used for any of the drugs, combinations of drugs or other agents described herein provided that the drugs, combinations of drugs or other agents maintain the ability to provide the function for which they are administered and that their activity can be confined to a desired site.

Transdermal Delivery

Although transdermal methods are generally considered to be a form of systemic delivery, the concentration of therapeutic agent will be highest at the site where delivery occurs and will decrease as the agents are distributed in the body. They may therefore, in some instances, be useful for the delivery of drugs or drug combinations described herein.

Transdermal delivery systems may optionally use minimally invasive technologies, such as iontophoresis, microneedles, electroporation, sonophoresis, and ultrasound to enhance drug delivery across the skin.

In delivery systems involving transdermal patches, drug combinations may be stored in a reservoir (reservoir type) or dissolved in a liquid or gel-based reservoir (matrix type). The starting point for the evaluation of the kinetics of drug release from a transdermal patch is an estimation of the drug compound's maximum flux across the skin (flux (J)) which is typically expressed in units of μg/cm²/h). Based on Fick's law of diffusion, the transport of therapeutic molecules across skin will be maintained until the concentration gradient ceases to exist. The permeability coefficient (P) can be obtained from the slope of a plot of cumulative permeation of diffusant vs. time obtained from an experimental permeation study.

A number of chemical and physical methods have been devised to enhance the delivery of drugs across the skin. For example, barrier properties of the stratum corneum may be altered by active/physical methodologies. Penetration enhancers, e.g., alcohols, sulphoxides, azone, pyrrolidones, essential oil, terpenes and terpenoids, fatty acids, water and urea may potentially be used for delivering the drugs and drug combinations described herein. However, a major limitation for penetration enhancers is that their efficacy is often closely correlated with the occurrence of skin irritation.

Semisolid vehicles such as proniosomes and microemulsion gels may also be used as penetration enhancers. Proniosomes are non-ionic based surfactant vesicles, that are known as “dry niosomes” because they may require hydration before drug release and permeation through the skin. Active methods for skin permeabilisation include ultrasound, electrically assisted methods (electroporation and iontophoresis), velocity based devices (powder injection, jet injectors), thermal approaches (lasers and radio-frequency heating) and mechanical methodologies such as microneedles (MN) and tape stripping. Active methods involve the use of external energy to act as a driving force for drug transport across the skin or by physically disrupting the stratum corneum.

Ultrasound Devices

Ultrasound is an oscillating sound pressure wave that has long been used in physics, chemistry, biology, and engineering in a wide range of frequencies. Ultrasound, sonophoresis, or phonophoresis can be defined as the transport of drugs across the skin by application of ultrasound perturbation at frequencies of 20 kHz-16 MHz, which has a sufficient intensity to reduce the resistance of skin. The use of ultrasound may potentially result in the effective delivery of the drugs and drug combinations described herein, by increasing skin permeability. The proposed mechanisms by which ultrasound effects tissues and cells include thermal effects and cavitation effects caused by collapse and acoustic streaming which can be explained as oscillation of cavitation bubbles in the ultrasound field. Ultrasound can increase the temperature of the insonated medium (the skin) by the absorption of the sound waves with a frequency greater than the upper limit of the human hearing range. Obviously, the higher the medium's absorption coefficient, the higher the increase in temperature and thus the greater the thermal effect. Cavitation is believed to be the predominant mechanism in the enhancement of TDD via ultrasound treatment.

Thermal Ablation

Thermal ablation is a method used to deliver drugs systemically through the skin by heating the surface of the skin, which depletes the stratum corneum selectively at the site of heating, without damaging deeper tissues. Radiofrequency (RF) thermal ablation involves the placement of a thin, needle-like electrode directly into the skin and application of high frequency alternating current (˜100 kHz) which produces microscopic pathways in the stratum corneum, through which drugs can permeate. Exposure of skin cells to a high frequency (100-500 kHz) causes ionic vibrations within the tissue which attempts to localize the heating to a specific area of the skin and thus ablate the cells in that region, resulting in drug transport across the skin. This technology may enable transdermal delivery of a wide variety of hydrophilic drugs and macromolecules using a low-cost, fully disposable device.

Microneedle Arrays

MN arrays are minimally invasive drug delivery systems that were developed to overcome some of the disadvantages commonly associated with hypodermic needle usage and in order to address and improve patient compliance. A wide variety of MN types and designs have been shown to be effective for the transdermal delivery of a diverse range of molecules, both in vitro and in vivo. A number of other physical approaches such as sonophoresis, electroporation, ultrasound and iontophoresis have also been combined with MN in order to enhance the permeation of drugs.

MN methods are efficacious, cost-effective and patient friendly. As a novel and minimally invasive approach, MN is capable of creating superficial pathways across the skin for small drugs, macromolecules, nanoparticles, or fluid extractions to achieve enhanced transdermal drug delivery. This method combines the efficacy of conventional injection needles with the convenience of transdermal patches, while minimizing the disadvantages of these administration methods.

### E. Specific Description of the Preferred Embodiment

In terms of specific embodiments, the invention is directed to an implantable drug delivery system for directionally delivering two or more different drugs (or combinations of drugs) comprising: a) a first section comprising a first therapeutic agent or combination of agents wherein, upon implantation into a patient, the first section directionally releases the first therapeutic agent or combination of agents over a period of time; and b) a second section comprising a second therapeutic agent or combination of agents wherein, upon implantation into a patient, the second section also directionally releases the second therapeutic agent or combination of agents over a period of time. The first section and second section of the delivery system are separated by a barrier that inhibits or blocks the passage of therapeutic agents between the first and second sections. Optionally the barrier may partly or completely cover surfaces of the gel other than the surface nearest or adjacent to the desired treatment or release site.

The implantable drug delivery system is preferably made of a hydrogel (e.g., made by 3-D printing and which, after implantation, may optionally dissolve over an extended period of time, e.g., 1-2 weeks, 1-4 weeks, 1-8 weeks, 1-12 weeks, etc.) with each section being separated by a polymeric barrier. When implanted, these therapeutic agents are released at a rate such that the dosage of each therapeutic agent is sufficient to make the combination therapeutically effective within a selected anatomic distance from the site of delivery (e.g., within 0.1-15 cm; 0.1-12, cm; 0.1-6.0 cm; 0.1-4.5 cm; 0.1-2.0 cm; 0.1-1.0 cm; 0.1-0.5 cm; 0.5-1.5 cm) but which does not have a substantial effect (e.g., does not substantially inhibit bone formation or calcification) outside of the selected anatomic distance. In an alternative embodiment, the amount or concentration of one or more released drugs at the furthest point of a given anatomic distance should be less than half of the amount or concentration at the nearest point. For example, the amount or concentration at 2.0 cm should be less that half of the amount or concentration at 0.1 cm.

In accordance with the drugs and drug combinations discussed herein and in US 2018/0071319, the implantable drug delivery system may have a first therapeutic agent or combination of therapeutic agents that promote bone or cartilage formation and a second therapeutic agent or combination of agents that prevent or treat heterotopic ossification, vascular calcification, or pathologic calcification. Any of the drug combinations described herein and any of the specific Hh pathway antagonists, statins and vitamin D related compounds may be used.

The agents that promote bone or cartilage formation may include one or more bone morphogenetic proteins with BMP-2 and BMP-7 being preferred. When implanted, these therapeutic agents are released at a rate such that the dosage of each therapeutic agent is sufficient to make the combination therapeutically effective at a treatment site where bone or cartilage formation is needed but which does not substantially promote bone formation or calcification outside of the treatment site. In contrast the agents that prevent or treat heterotopic ossification, vascular calcification, or pathologic calcification are release at a site away from the site where bone or cartilage formation is needed and into tissue.

The invention also includes a method for treating a patient that has undergone orthopedic surgery resulting in an anatomic treatment site where bone or cartilage formation is needed, or who has undergone trauma resulting in at least one anatomic treatment site comprising a broken bone or damaged cartilage. The method comprises implanting into the patient the implantable drug delivery system described above. The delivery system should be positioned in close proximity, or adjacent to, the anatomic treatment site and should be oriented so that the section comprising the first therapeutic agent or combination of therapeutic agents is closest to the anatomic treatment site and releases therapeutic agents that promote bone formation at that site. The second section comprising the second therapeutic agent or combination of therapeutic agents should be opposite to, or otherwise removed from, the anatomic treatment site and release therapeutic agents that prevent or treat heterotopic ossification, vascular calcification, or pathologic calcification in tissues adjacent to, or in close proximity to, the second section. Among the most severe problems associated with orthopedic surgery are infections and osteoarthritis. Thus, the use of antibiotics and anti-inflammatories into tissues, with or without agents that inhibit unwanted calcification or bone formation, will often be desirable. These agents should generally be gradually released for at least a week, and depending on clinical factors, at least two weeks, 4 weeks, 6 weeks, 8 weeks or longer, after surgery, preferably from a gel that dissolves concurrently with, or soon after the completion of, release.

Other drugs, prophylactic agents, analgesics, anesthetics, carriers, saline, buffers or other agents may be used together with, or in the place of the first therapeutic agent or combination of therapeutic agents and/or in the place of the second therapeutic agent or combination of therapeutic agents. Specific agents may be those discussed above in connection with the treatment of broken bones, or cytotoxic agents, toxins, chemotherapeutic agents, anti-cancer or anti metastasis agents, hormones (e.g., growth hormone), etc.

All of the drugs and drug combinations described herein, may advantageously be formulated as small particles, e.g., nanoparticles, which should aid in their diffusion and distribution from devices.

### F. Formulating Drugs

The compounds described herein may be administered to patients in a pharmaceutical composition comprising the compound along with a pharmaceutically acceptable carrier. The carrier may be any solvent, diluent, liquid or solid vehicle that is pharmaceutically acceptable and typically used in formulating drugs compatible with implantable delivery. Guidance concerning the making of pharmaceutical formulations can be obtained from standard works in the art (see, e.g., Remington's Pharmaceutical Sciences, 16th edition, E. W. Martin, Easton, Pa. (1980)). In addition, pharmaceutical compositions may contain any of the excipients that are commonly used in the art.

Emulsions

Solvent emulsification diffusion (SED) is a commonly used method for the preparation of solid-lipid and polymeric nanoparticles and may potentially be used with the therapeutic combinations and therapeutic devices described herein. The ease and convenience in fabrication of nanoemulsions can precisely control drug loading and positioning (spatial placement) which is an important consideration for the delivery of the drugs and drug combinations described herein for the treatment or prevention of heterotopic ossification, vascular calcification, or other pathologic calcification.

Supercritical Fluids in Nanoparticle Production

Supercritical fluid techniques may also potentially be important in fabricating the desired formulations for the delivery of the drugs and drug combinations described herein. SC (supercritical) CO₂ is commonly used as a supercritical fluid. Above its supercritical point, CO₂ can serve as a solvent with low density, and this unique property may be used to increase nanoparticle production from a lab scale to pilot scale. One of the most popular supercritical fluid techniques—the rapid expansion of supercritical solution (RESS)—may potentially be used in the formulation of the present combinations. RESS can produce nanoparticles with a diameter of less than 100 nm and free of solvents, which is important for the fabrication of nanosuspensions. The combination of a supercritical fluid with an emulsion process offers greater benefit than the use of a single technique alone.

Nanomedicinal Formulations (Including Liposomes)

Nanomedicinal formulations are nanometer-sized carrier materials designed for increasing the drug tissue bioavailability. Two approaches may be used for the production of nanoscale formulations, a top-down approach, and a bottom-up approach. Bottom-up techniques include chemical reactions and molecular assemblies, such as supercritical fluid techniques, precipitation, nanoemulsion, spray-drying, polymerization and synthesis. Apart from classical synthesis and polymerization, one powerful bottom-up technique is the formation of nanoemulsions.

A top-down approach may include wet media milling and high-pressure homogenization. The resulting nanoparticles are nanosuspensions, stabilized submicron colloidal dispersions of nanosized drug particles. The bioavailability associated with preparations may be increased using high-pressure homogenization or wet ball milling.

Protein Carriers

Proteins are often a first choice for developing nanoscale drug formulations because of their safety, biocompatibility and ease of availability. Most proteins are nanoscale macromolecules and hydrophilic or hydrophobic sequences in the proteins can be loaded with different drugs. Albumin has been extensively explored because it is the most abundant protein in human serum and is extremely stable during bioprocessing and in pharmaceutical preparations compared to the other proteins. Most importantly, it can accumulate in tissues and inflammation sites and is therefore a good choice for passive targeted drug delivery.

### G. Dosage

The dosage administered to a patient will vary depending on the particular agents being administered, the disease or condition being treated, the route of administration and clinical factors unique to the individual being treated. Where administration is to treat or prevent heterotopic ossification, vascular calcification, or other pathologic calcification, it is generally expected that patients will receive 0.1-500 mg/day (typically 0.5-500 mg/day) of an Hh pathway antagonist; 0.3-3000 IU/day (typically 100-3000 IU/day) of vitamin D, cholecalciferol or a vitamin D analog; and/or 0.1-500 mg/day (typically 0.5-500 mg/day) of a statin. These example dosages apply regardless of whether a patient is administered: a) an Hh pathway antagonist in combination with vitamin D, cholecalciferol or a vitamin D analog but without a statin; b) an Hh pathway antagonist in combination with a statin but without vitamin D, cholecalciferol or a vitamin D analog; or c) an Hh pathway antagonist in combination with vitamin D, cholecalciferol or a vitamin D analog and a statin. Of these possibilities, it is the combination containing all three agents that is most preferred.

The dosages of other drugs delivered using the systems described herein will be based on recommendations in the art on the dosages for such drugs when delivered locally or, in cases where recommendations are not available, will be determined using standard pharmacological methods.

### H. Potential of Synergism for Combinations of Drugs

Overall, particularly preferred methods of preventing or treating heterotopic ossification, vascular calcification, or other pathologic calcification involve the administration of: a) an Hh pathway antagonist selected from the group consisting of: zerumbone epoxide; staurosporinone; 6-hydroxystauro-sporinone; arcyriaflavin C; 5,6-dihyroxyarcyria-flavin A; physalin F; physalin B; cyclopamine; HPI-1, HPI-2; HPI-3, or HPI-4; arsenic trioxide; sodium arsenite; phenylarsine; GANT-58; GANT-61; zerumbone; and inhibitors of the expression of the genes Ptch1, Gli1 or HIP, with the most preferred being arsenic trioxide; b) vitamin D or cholecalciferol; and c) a statin selected from the group consisting of Atorvastatin; Fluvastatin; Pravastatin; Rosuvastatin; Simvastatin; Pitavastatin; Cerivastatin; Lovastatin; and Mevastatin. The dosage of each compound should be sufficient to make the overall treatment therapeutically effective. It may also potentially be possible to obtain a synergistic effect. In the context used herein, the term “synergistic” means that the effect of a combination of drugs is greater than the maximum effect that can be achieved when the drugs are used individually. For example, the combination of ATO, cholecalciferol and lovastatin would be acting synergistically with respect to preventing heterotopic ossification after surgery if, compared to an untreated group, fewer people developed ectopic ossification when given the combination then when administered any one component of the combination alone. When used to treat existing ectopic ossification, synergism could potentially occur with respect to one or more symptoms associated with ossification such as pain, swelling, range of joint motion etc. In another context, the term “synergistic” may refer to a reduction in the number of patients experiencing side effects or the severity of the side effects when a combination of drugs is administered compared to when the drugs are used individually. Thus, synergism may refer to an improvement in the safety of drugs. Synergism could also potentially manifest itself in other ways, such as the rapidity with which relief from a symptom is first experienced or the duration of action.

### I. Treatment Methods

With respect to the treatment or prevention of heterotopic ossification, vascular calcification, and pathologic calcification, subjects undergoing treatment according to the methods described herein will generally fall into two categories. The first consists of individuals that do not yet have abnormal bone formation or calcification but are part of a group recognized as being prone to this occurring. Included in this group are patients that have undergone surgery (particularly arthroscopic surgery of a hip or other joint), and those that have undergone traumatic injuries, fractures, wounds, head or brain injuries and burns. The group also includes subjects with atherosclerosis, that have had a myocardial infarction or that have a genetic disease associated with ectopic bone formation or calcification. The objective in these cases is to reduce the likelihood of HO or abnormal calcification occurring. In general, these patients will continue treatment until the attending physician is satisfied that increased risk has subsided. This may be anywhere from a few weeks up to several years. In the case where increased risk of heterotopic ossification, vascular calcification, or pathologic calcification is due to genetic factors or ongoing disease, administration may be continued for the life of the patient.

The second category of patients will be those that have been identified clinically as already suffering from heterotopic ossification, or pathologic calcification and for whom the objective is primarily to treat the existing condition. In general, these patients will be administered compositions in the same manner as those in which the objective is prevention but dosages and dosing schedules may be varied depending on the degree to which a response is observed and may be combined with physical therapy or surgery.

However, the invention is not limited to the treatment of heterotopic ossification, vascular calcification, and pathologic calcification and, more generally, encompasses any disease or condition and any treatment method using one of the devices described herein. Included in this are autoimmune diseases, cancers, inflammatory diseases, respiratory diseases, diseases of the cardiovascular system; neurological diseases and renal diseases. In all cases, treatment methods will be selected by physicians based on clinical factors unique to individual patients.

All references cited herein are fully incorporated by reference. Having now fully described the invention, it will be understood by one of skill in the art that the invention may be performed within a wide and equivalent range of conditions, parameters and the like, without affecting the spirit or scope of the invention or any embodiment thereof.

1. A method for inhibiting osteogenesis in mesenchymal stem cells in a patient, comprising contacting said mesenchymal cells with a drug selected from the group consisting of a Hedgehog (Hh) pathway antagonist; vitamin D, cholecalciferol or a vitamin D analog and a statin; or a combination of drugs selected from:
a) a combination of an Hedgehog (Hh) pathway antagonist together with:
i) vitamin D, cholecalciferol or a vitamin D analog; or
ii) a statin;
b) a combination of:
i) vitamin D, cholecalciferol or a vitamin D analog; and
ii) a statin; or
c) a combination of:
i) an Hh pathway antagonist;
ii) vitamin D, cholecalciferol or a vitamin D analog; and
iii) a statin;
wherein said drug or combination of drugs of paragraphs a), b) and c) are administered to the patient by:
aa) localized delivery using: implanted or topically applied hydrogels, poloxamer gels, polysaccharide gels; nanomedicinal formulations; 3D printed gels; or microemulsions;
bb) 3D printed formulations or encapsulated drugs;
cc) nanoscale drug delivery systems using liposomes and nanoparticles;
dd) a microneedle array; and
ee) transdermal delivery or implantable sponges soaked in drugs;
and wherein said drug or combination of drugs is at a dosage sufficient to make the drug or combination of drugs effective at inhibiting osteogenesis in mesenchymal stem cells within a selected anatomic distance from the site of delivery but which does not substantially inhibit osteogenesis in mesenchymal stem cells outside of the selected anatomic distance.

2. The method of claim 1, wherein said method is used to prevent or treat heterotopic ossification subsequent to spinal cord damage, traumatic injury, head or brain injuries, burns, bone fractures, muscle injuries, or joint replacement surgery.

3. The method of claim 1, wherein said method is used to prevent or treat myositis ossificans; progressive osseous heteroplasia, fibrodysplasia ossificans progressiva or Albright's hereditary osteodystrophy.

4. The method of claim 1, wherein said method is used to prevent or treat myositis ossificans or fibrodysplasia ossificans progressiva in a cat or dog.

5. The method of claim 1, wherein said Hh pathway antagonist is administered to said patient at 0.5-500 mg/day; vitamin D, cholecalciferol or a vitamin D analog is administered at 100-3000 IU/day; and said statin is administered at 0.5-500 mg/day.

6. The method of claim 1, wherein said Hh pathway antagonist is a ligand that binds to the Sonic receptor and prevents activation; an antibody that binds to either Sonic, Desert or Indian or to the receptor for these ligands; or an siRNA.

7. The method of claim 1, wherein said Hh pathway antagonist is selected from the group consisting of: a) zerumbone epoxide; b) staurosporinone; c) 6-hydroxystauro-sporinone; d) arcyriaflavin C; e) 5,6-dihyroxyarcyriaflavin A; f) physalin F; g) physalin B; h) cyclopamine; i) HPI-1, HPI-2; HPI-3; or HPI-4; j) arsenic trioxide (ATO); k) sodium arsenite; l) phenylarsine; m) GANT-58; n) GANT-61; o) zerumbone; and p) inhibitors of the expression of the genes Ptch1, Gli1 or HIP.

8. The method of claim 7, wherein said Hh pathway antagonist is arsenic trioxide (ATO) administered to said patient at a dosage of between 0.05 to 0.20 mg/kg/day.

9. The method of claim 1, wherein one or more drugs are encapsulated and/or in the form of nanoparticles.

10. The method of claim 1, wherein said statin is selected from the group consisting of: Atorvastatin; Fluvastatin; Pravastatin; Rosuvastatin; Simvastatin; Pitavastatin; Cerivastatin; Lovastatin; and Mevastatin.

11. A method for preventing or treating heterotopic ossification, vascular calcification, or pathologic calcification in a patient, comprising administering to said patient a drug selected from the group consisting of a Hedgehog (Hh) pathway antagonist; vitamin D, cholecalciferol or a vitamin D analog and a statin; or a combination of drugs selected from:
a) a combination of a Hedgehog (Hh) pathway antagonist together with:
i) vitamin D, cholecalciferol or a vitamin D analog; or
ii) a statin;
b) a combination of:
i) vitamin D, cholecalciferol or a vitamin D analog; and
ii) a statin; or
c) a combination of:
i) an Hh pathway antagonist;
ii) vitamin D, cholecalciferol or a vitamin D analog; and
iii) a statin;
wherein said drug or combination of drugs of paragraphs a), b) and c) are administered to the patient by:
aa) localized delivery using: implanted or topically applied hydrogels, poloxamer gels, polysaccharide gels; nanomedicinal formulations; 3D printed gels; or microemulsions;
bb) 3D printed formulations;
cc) nanoscale drug delivery systems using liposomes and nanoparticles;
dd) a microneedle array; and
ee) transdermal delivery or implantable sponges soaked in drugs;
and wherein the drug or combination of drugs is at a dosage sufficient to make the drug or combination of drugs effective at preventing or treating heterotopic ossification, vascular calcification, or pathologic calcification within a selected anatomic distance from the site of delivery but which does not substantially inhibit bone formation or calcification outside of the selected anatomic distance.

12.-26. (canceled)

27. An implantable drug delivery system for directionally delivering two or more different drugs comprising:
a) a first section comprising a first therapeutic agent or combination of therapeutic agents wherein, upon implantation into a patient, said first section releases said first therapeutic agent or combination of therapeutic agents over a period of time;
b) a second section comprising a second therapeutic agent or combination of therapeutic agents wherein, upon implantation into a patient, said second section releases said second therapeutic agent or combination of agents over a period of time;
wherein said first section and said second section are separated by a barrier that inhibits or blocks the passage of therapeutic agents between the first and second sections and which limits the area in which the first and second therapeutic agents are released.

28. The implantable drug delivery system of claim 27, wherein said first and second sections comprise polymeric gels separated by a barrier that is impermeable to the first therapeutic agent or combination of therapeutic agents and that inhibits or is impermeable to the second therapeutic agent or combination of therapeutic agents.

29. The implantable drug delivery system of claim 28, wherein said first and second sections comprise hydrogels with drugs or drug combinations interspersed or compartmentalized in the hydrogels.

30. The implantable drug delivery system of claim 28, wherein said first and second sections comprise one or more polymeric layers with drugs or drug combinations interspersed or compartmentalized in the gel and with the barrier that is impermeable to drugs or combinations of drugs separating layers comprising the first therapeutic agent or combination of therapeutic agents from layers comprising the second therapeutic agent or combination of therapeutic agents.

31. The implantable drug delivery system of claim 28, wherein the gels are provided with an amount of drug or combination of drugs such that, when the drug delivery system is implanted, each drug or combination of drugs is released at a rate that results in a dosage sufficient to be therapeutically effective only within a selected anatomic distance from the site of delivery.

32. (canceled)

33. The implantable drug delivery system of claim 27, wherein one section comprises a therapeutic agent or combination of therapeutic agents that promotes bone formation and/or one section comprises a therapeutic agent or combination of therapeutic agents that prevent or treat heterotopic ossification, vascular calcification, or pathologic calcification.

34. (canceled)

35. The implantable drug delivery system of claim 27, wherein a first section that optionally comprises a first therapeutic agent or combination of therapeutic agents and a second section with a second therapeutic agent or combination of therapeutic agents, wherein the second therapeutic agent or combination of therapeutic agents prevent or treat heterotopic ossification, vascular calcification, or pathologic calcification.

36. The implantable drug delivery system of claim 35, wherein the second section comprises a therapeutic agent is selected from the group consisting of a Hedgehog (Hh) pathway antagonist, vitamin D, cholecalciferol, a vitamin D analog and a statin; or a combination of drugs selected from:
a) a combination of a Hedgehog (Hh) pathway antagonist together with:
i) vitamin D, cholecalciferol or a vitamin D analog; or
ii) a statin;
b) a combination of:
i) vitamin D, cholecalciferol or a vitamin D analog; and
ii) a statin; and
c) a combination of:
i) an Hh pathway antagonist;
ii) vitamin D, cholecalciferol or a vitamin D analog; and
iii) a statin.

37. (canceled)

38. The implantable drug delivery system of claim 35, wherein, when implanted, the implantable drug delivery system releases therapeutic agents at a rate that results in a dosage sufficient to be therapeutically effective only within a selected anatomic distance from the site of delivery.

39.-56. (canceled)
